(19) United States
(12) Patent Application Publication
(10) Pub. No.: US 2026/0367032 A1
(43) Pub. Date: Nov. 19, 2026

(54) MONOVALENT ION SELECTIVE ELECTRODE SENSORS, MEMBRANE COMPOSITIONS, AND METHODS TO REDUCE BENZALKONIUM INTERFERENCE FOR DIAGNOSTIC ANALYZERS

(71) Applicant: Siemens Healthcare Diagnostics Inc., Tarrytown, NY (US)

(72) Inventor: Wei Zhang, Needham, MA (US)

(73) Assignee: Siemens Healthcare Diagnostics Inc., Tarrytown, NY (US)

(21) Appl. No.: 19/133,952

(22) PCT Filed: Nov. 10, 2023
(86) PCT No.: PCT/US2023/079332

§ 371 Date: May 29, 2025

Related U.S. Application Data
(60) Provisional application No. 63/385,818, filed on Dec. 2, 2022.

(51) Int. Cl. G01N 27/333

(52) U.S. Cl. CPC G01N 27/3335

(57) ABSTRACT

A polymeric ion selective electrode (ISE) sensor for use in blood gas analyzers for blood gas testing, as well as polymeric membranes for same, are disclosed. The polymeric ion selective electrode sensor comprises a polymeric membrane that is selective for a monovalent cation and that has a chemical composition containing a boron-containing salt and at least one monovalent selective ionophore. Also disclosed are polymeric ion selective membranes and methods of minimizing benzalkonium (BK) interference in an ion selective electrode sensor are provided, as are other aspects.

This application claims benefit under 35 USC § 119(e) of U.S. Provisional Application No. 63/385,818, filed Dec. 2, 2022. The entire contents of the above-referenced patent application are hereby expressly incorporated herein by reference.

### TECHNICAL FIELD

This disclosure relates to ion selective electrode sensors, polymeric ion selective membranes, polymeric membrane chemical compositions, and methods of reducing interference in blood gas testing performed by diagnostic analyzers.

### BACKGROUND

Polymeric membrane-based ion selective electrode (ISE) sensors are used to measure various blood gasses using blood gas analyzers. For example, blood gases such as Na+, K+, Ca++, Mg++pH, PCO₂, and PO₂ may be quantified. An ISE sensor is a transducer that converts the activity of a specific target cation present in a liquid into measurable electrical potential.

In order to keep a blood gas analyzer functioning properly, such blood gas analyzers containing the ion selective membranes (ISMs), can undergo routine cleaning procedures from time-to-time, to clean off blood and other contamination from the blood gas analyzer. One common sanitary cleaning agent used to clean such blood gas analyzers is a benzalkonium-containing cleaning agent (hereinafter “BK-containing cleaning agent”), such as benzalkonium chloride. Benzalkonium chloride is classified as a quaternary ammonium antiseptic and disinfectant. Such BK-containing cleaning agents can sometimes come into contact with the ISMs as a consequence of the cleaning. If sufficiently contacted and exposed, the BK component of the cleaning agent can impact test results.

In some cases, the impact is small enough that it can cause an automated recalibration adjustment, which then can produce a bias offset to account for small BK interferences. However, in some instances, the BK interference can be so large that it can cause delay in the use of the blood gas analyzer after such cleaning until such time that the interference falls below a baseline threshold.

### BRIEF DESCRIPTION OF THE DRAWINGS

FIG. 1 is a schematic diagram of a polymeric membrane-based ion selective electrode (ISE) sensor in a sensing apparatus according to certain non-limiting embodiments of the disclosure.

FIG. 2A is a bottom view of a cartridge including one or more polymeric membrane-based ion selective electrode (ISE) sensors according to certain non-limiting embodiments of the disclosure.

FIG. 2B is a cross-sectioned side view of another polymeric membrane-based ion selective electrode (ISE) sensor within a cartridge according to certain non-limiting embodiments of the disclosure.

FIG. 2C is a top view of a diagnostic cartridge including a few polymeric membrane-based monovalent ion selective electrode (ISE) sensors according to certain non-limiting embodiments of the disclosure.

FIG. 3 is a flowchart depicting a method of minimizing sensor interference in an ion selective electrode sensor according to certain non-limiting embodiments of the disclosure.

### DETAILED DESCRIPTION

Independent of the grammatical term usage, individuals with male, female or other gender identities are included within the term.

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, and/or methods disclosed herein can be made and executed without undue experimentation in light of the present disclosure. While the articles, compositions, kits, and/or methods have been described in terms of particular embodiments, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

In some non-limiting embodiments, a polymeric ion selective electrode sensor is provided. The polymeric ion selective electrode sensor comprises a polymeric membrane selective for monovalent cations having a chemical composition containing a boron-containing salt present at a concentration in a range of from about 0.6 wt % to about 1.8 wt % (weight percentage), based on the total weight of all non-solvents in the chemical composition. Such polymeric ion selective electrode sensors mitigate benzalkonium (BK) interference in monovalent cation measurements (e.g., Na+, K+) taken by the blood gas analyzers they are used in.

In other non-limiting embodiments, a chemical composition of matter of a polymeric ion selective membrane is provided. The chemical composition of the ion selective membrane comprises a polymer such as polyvinyl chloride, a boron-containing salt provided at a concentration in a range of from about 0.6 wt % to about 1.8 wt %, based on the total weight of all non-solvents in the chemical composition, a plasticizer, and at least one monovalent selective ionophore.

In yet a further non-limiting embodiment, a method of minimizing sensor interference in an ion selective electrode sensor is provided. The method comprises providing an ion selective electrode sensor comprising an ion selective membrane, the ion selective membrane having a chemical composition further comprising: a polymer, a boron-containing salt at a concentration in a range of from about 0.6 wt % to about 1.8 wt %, based on the total weight of all non-solvents in the chemical composition, a plasticizer, and at least one monovalent selective ionophore, and exposing the ion selective membrane to a benzalkonium-containing cleaner, wherein the ion selective electrode sensor has less than about +10.0% induced bias offset upon exposure to the benzalkonium-containing cleaning agent (for example, but not by way of limitation, a benzalkonium-containing cleaning agent having a benzalkonium concentration of at least about 25 μg/mL).

Numerous other aspects are provided in accordance with the disclosure. Other features and aspects of the present disclosure will become fully apparent from the following detailed description, the claims, and the accompanying drawings.

In diagnostic analyzers, such as blood gas analyzers, polymer-based (e.g., polyvinyl chloride (PVC)) ion selective electrode sensors are used having a polymeric membrane as the ion selective element. Such polymeric membranes can be designed to be selective to certain cations, such as monovalent cations (e.g., Na+, K+) and multivalent cations (e.g., Ca++, Mg++). Periodically, or as needed, such blood gas analyzers are cleaned using a cleaning agent for hygiene and sterilization purposes. In particular, benzalkonium (BK)-containing cleaning agents are often used to clean these diagnostic analyzers. During and after such cleaning, the polymeric membrane of such ion selective electrode (ISE) sensors can become/be exposed to the BK-containing cleaning agent. Such exposure of the polymeric membrane to such BK-containing cleaning agents can result in significant interference with the ion selective membranes. This is especially true of monovalent ion selective membranes, such as those specifically selective for Na+ and K+ cations. The more the exposure to the BK-containing cleaning agent, the more the test results can be affected.

For example, slight exposure can cause offset bias of reporting results. In some cases, the bias is small enough that it can be corrected through a recalibration adjustment. In more severe exposure cases, the contamination can result in reporting an error value. Such contamination can even cause a temporary shutdown of the blood gas testing on the blood gas analyzer to allow time for the ion selective membrane sensor to get back to a pre-established or control baseline reading.

This BK interference can occur in analyzers that quantify blood gas target ions with an ISE sensor comprising a polymeric membrane that includes ionophores specific to specific target ions. Inventors herein have found that the polymeric membrane selective for monovalent cations can exhibit significant interference after undergoing exposure to a BK-containing cleaning agent. Among these, polymer membranes that target the monovalent cation Na+ can have a very substantial impact from BK exposure, while interferences with target monovalent K+ ions can also be significant. When an ISE sensor configured to target Na+ is affected by BK interference, the Na+ slope measurement can often be out of specification (e.g., low).

Once the ISE sensor is substantially affected by such BK interference, it sometimes takes a significant amount of time (e.g., 2 hours or one day or more) to get back to an acceptable baseline reading for normal performance, so that the monovalent cation quantification can again take place. Accordingly, the ISE sensor may not be available for running testing for certain cations for hours, which effectively can make the blood gas analyzer less useful. Moreover, this wait time can be a cause of field complaints by users of such blood gas analyzers.

Among the various ISE sensors, the monovalent ISE sensors, and specifically those targeting monovalent Na+ cations, were found to be the most sensitive to BK interference due to their high concentration in the physiological solution (approximately 100 mM to 180 mM Na+) comparing to K+ monovalent electrolytes (e.g., 4.0 mM K+). The bias specification for Na+ is relatively tighter than for K+. For an RP500 analyzer available from Siemens-Healthineers having an ion selective sensor, when BK interference is detected (based on a P-Wash/B-Wash signal difference), a recalibration sequence (Benzalkonium recalibration) is triggered. With this recalibration sequence, the ISE sensor's offset value can be compensated for in an attempt to make-up/offset for the sample signal shift due to BK interference and thus bring the test back within proper bounds.

However, this solution of offsetting for BK interference is less than ideal. For example, in some cases the recalibration mode cannot fully compensate the offset shift and thus the ISE sensor can still be out of allowable specification (outside of an allowable offset bias). Currently, no effective approach has been applied to solve the readiness delay of monovalent Na+ (and K+ ions) when a BK interference event occurs. Using prior art analyzers with Na+ selective ISE sensors, the operator simply has to wait until the slope and offset drift return to within acceptable specifications before Na+ testing can again take place.

Benzalkonium interference can be contributed by multiple factors, such as membrane perm-selectivity, lipophilic anionic sites, ionophore-analyte binding constant, plasticizer content, etc. However, among these, the inventors hereof have discovered that the borate weight percentage (wt %) content in the chemical composition of the polymer membrane plays a substantial role in resisting BK interference. Wt % as used herein for borate is based on 100% of the non-solvents in the composition (i.e., excluding any solvents). For example, a lipophilic boron-containing salt (lipophilic borate) can be compounded into the polymer membrane (e.g., PVC membrane) to manipulate charge balance at the phase boundary and also mass balance at phase boundary with its ion-exchanging feature (e.g., Hofmeister series, which ranks ion selectivity ability). Lipophilic boron-containing salts, such as (but not limited to) potassium tetrakis (4-chlorophenyl) borate (KTpClPB) and/or sodium tetrakis[3,5-bis(trifluoromethyl)phenyl]borate (NaTFPB), may be used.

Not to be bound by theory, it is possible that when low borate sites (e.g., about 0.2 wt % borate or less) are present in ISE membrane, positively charged Benzalkonium ions are inclined to interact with ionophore so that the sensor loses its recognition capability to the target monovalent cation (e.g., Na+, K+). Then such plasticized membrane surface is converted to provide a pro ion-exchange response to level of benzalkonium contamination. With appropriate borate content in the membrane, it forms an electrostatic association with sample benzalkonium cation (could be ion pairing or a more loose association). This may lead to reduction of benzalkonium cation contribution to membrane surface ion-exchange process, which competes with the response process of ionophore interaction to the target ion (e.g., Na+ ion).

Likewise, when the borate content is too high (e.g., >2.0 wt %), it is believed that the sensor membrane response selectivity over other co-existing cations can also be changed preferring Hofmeister series (e.g., preferring K+>Na+>Ca2+). Na+ sensor response sensitivity to Na+ can be compromised due to co-existing electrolytes including cations and anions. Also when too much borate is added, its solubility in the plasticizer may exceed the threshold and there may be borate salt exudation. In circumstances of BK free condition, borate to ionophore ratio plays a substantial role in other essential sensing functions (e.g., selectivity over co-existing electrolytes, response speed, initial wet up period, etc.).

To improve the ISM sensor's resistance to benzalkonium interference and also the ISM sensor's essential response function, borate-to-ionophore ratio as well as borate content in ISM can both be considered in the ISM formulation.

In view of the above expressed issues and concerns, polymeric ISE sensors, ion-selective polymeric membranes, and methods of minimizing BK interference in ion selective sensors are provided. According to certain non-limiting embodiments, the potentiometric measurements of monovalent electrolytes such as Na⁺ and K+, for example, can be carried out by the ISE sensor embodied in a blood gas analyzer comprising an ion-selective membrane (ISM) containing a boron-containing salt. The range of boron-containing salt wt % to achieve improvements in BK interference was discovered to be in a range of from about 0.6 wt % to about 1.8 wt % boron-containing salt, between about 0.7 wt % and about 1.7 wt % boron-containing salt in some non-limiting embodiments, or even between about 0.9 wt % and about 1.6 wt % in other non-limiting embodiments, all based on the total weight of all non-solvents in the chemical composition.

Polymeric ISE sensors, ion-selective polymeric membranes, and methods of the disclosure offer substantially lowered BK interference when selective for monovalent Na+ and K+ cations. The inventive ISMs comprise a plasticized polymer doped with target monovalent selective ionophore(s) and a boron-containing salt in the above-listed wt %. The ISM including boron-containing salt in the defined wt % further comprises a polymeric compound (e.g., PVC) that can selectively bind to the monovalent ions of interest (e.g., Na+, K+) via coordinate bonds.

The ISM can be prepared by mixing a polymeric phase such as polyvinyl chloride (PVC) with a suitable plasticizer. In embodiments that are selective to Na+ ions, in order to make this PVC/plasticizer material sensitive to Na⁺ ions, it can be doped with highly hydrophobic sodium (Na+) ionophore(s). Likewise, the PVC/plasticizer material sensitive to K⁺ ions can be doped with potassium (K+) ionophore(s). Both Na+ ionophores and K+ ionophores are non-ionic molecules that can selectively chelate Na+ and K+, respectively.

Thus, in accordance with one non-limiting aspect, a membrane chemical composition of matter (herein “membrane composition”) is provided that can be used in an ISE sensor to selectively measure monovalent cations, such as Na+ and K+. In particular, the membrane composition of the membrane has improved resistance to detection loss due to exposure to BK, such as from a BK-containing cleaning agent. The improved membrane composition can be used to form a polymeric ISM in such ISE sensors to achieve reduced induced bias, and thus also reduced magnitudes of offset correction. For Na+ detection, shutdown of the blood gas analyzer due to BK interference can be largely mitigated.

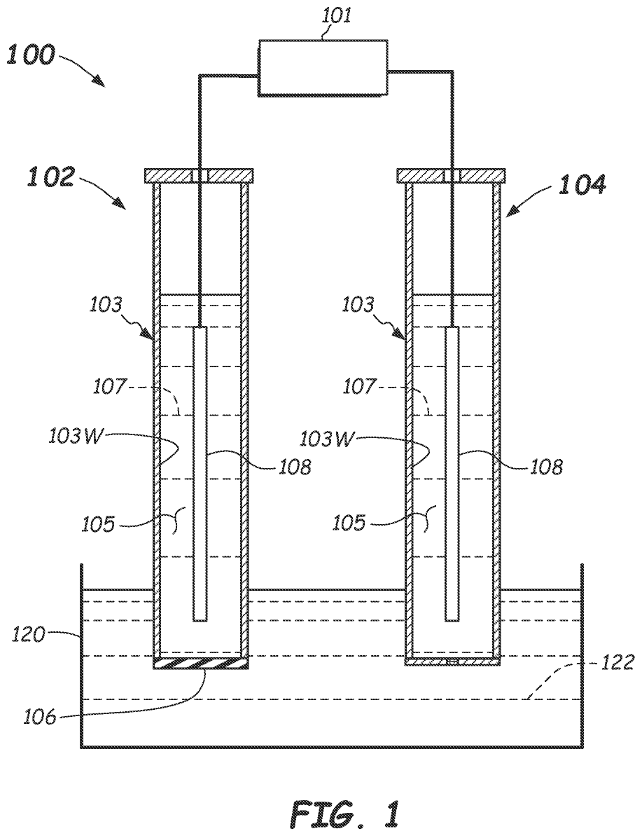

Further details and examples of polymeric membrane chemical compositions, polymeric ISE membranes, polymeric ISE sensors, and methods of the disclosure are provided with reference to FIGS. 1-3 herein.

Referring now to FIG. 1, an example of a measurement system 100 (hereinafter system 100) is shown. System 100 can include an indicator electrode, which is described as the ion selective electrode sensor 102 herein, and a reference electrode 104. The ion selective electrode sensor 102 and the reference electrode 104 can be electrically coupled to a detection system 101, which may include any suitable electronics to enable reading an electrical potential (or current) difference between the ion selective electrode sensor 102 and the reference electrode 104 as a measurable signal. In the case of a potential difference, the detection system 101 can include a potentiometer. In the case of a current difference, the detection system 101 can include an ammeter. The detection system 101 and the reference electrode 104 construction are well known and will not be described further herein.

In more detail, the ion selective electrode sensor 102 comprises an electrode body 103, which may be any suitable insulator material, having an ion selective membrane (ISM) 106 located at its lower end, for example. The walls 103W of the electrode body 103 and the ISM 106 form a reservoir 105. The ISM 106 can be formed as a thin polymer sheet that is selective to certain monovalent cations, such as Na+ and K+ monovalent cations described herein. In short, the ISM 106 functions to allow the selective cations (e.g., Na+ or K+ cations depending on the ISM design) to pass through the ISM 106, while not allowing other non-selected cations to pass through the ISM 106. The chemical composition of the ISM 106 making it selective for either Na+ cations or K+ cations is fully described herein.

Attachment of the ISM 106 to the electrode body 103 may be by any suitable means, such as bonding, compression and sealing using a sealing ring, or other suitable attachment means for sealing the membrane-body interface. The internal reservoir 105 formed by walls 103W and ISM 106 can be filled with a suitable electrolyte solution 107. Electrolyte solution 107 can be a salt solution, such as NaCl or KCl or a mixture of multi-electrolyte salt solution, for example. However, other suitable electrolyte solutions or gels (e.g., hydrogels) may be used, such as (but not limited to) polyvinyl alcohol (PVA), methocel, and/or methacrylamidopropyltrimethylammonium chloride (MAPTAC).

Within the reservoir 105 and in contact with the electrolyte solution 107, an electrode 108 can be included. Electrode 108 can be of any suitable construction, such as coated metal rod shown. For example, a silver (AG) rod can be coated with a silver chloride (AGCl) coating, as is known in the art, for example. However, the electrode 108 may optionally or additionally be made from gold, platinum, or the like. To condition the ion selective electrode sensor 102, it may be immersed in a conditioning solution with a similar concentration of the analyte to be measured. For example, an Na+ selective sensor 102 may be conditioned through immersion in a conditioning solution having Na+ concentration of from about 100 mM to about 150 mM, for example. A K+ selective sensor 102 may be conditioned through immersion in a conditioning solution having K+ concentration of from about 3 mM to about 5 mM, for example.

Once conditioned, the system 100, including the ion selective electrode sensor 102 and reference sensor 104 may be operated to detect a potential (or current) difference by immersing the sensors in a container 120 including the sample 122 or otherwise exposing the ion selective electrode sensor 102 and reference sensor 104 to the sample 122. The container 120 can be a vat, groove, channel, passageway, or like providing intimate contact between the sample 122 and the membrane 106 of the ion selective electrode sensor 102 as well as with the reference sensor 104. In some non-limiting embodiments, the ion selective electrode sensor 102 and reference sensor 104 may be included in, or in measuring contact with, a passageway that has whole blood as the sample 122 contained therein or passing there through. The sample 122 may be injected or otherwise flowed into the passageway, for example. As was discussed above, the ion selective electrode sensor 102 includes a novel membrane chemical composition that can mitigate BK interference thereof.

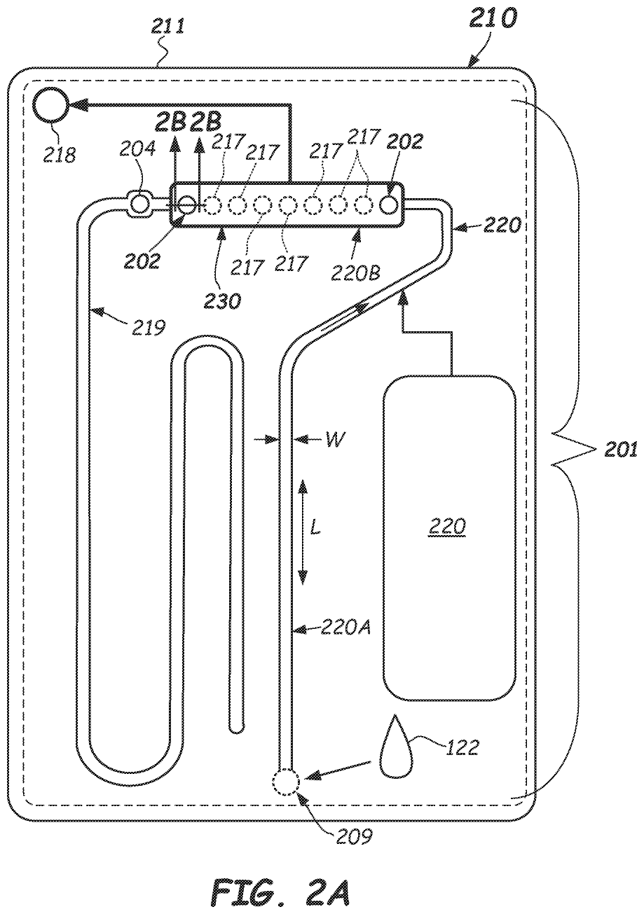

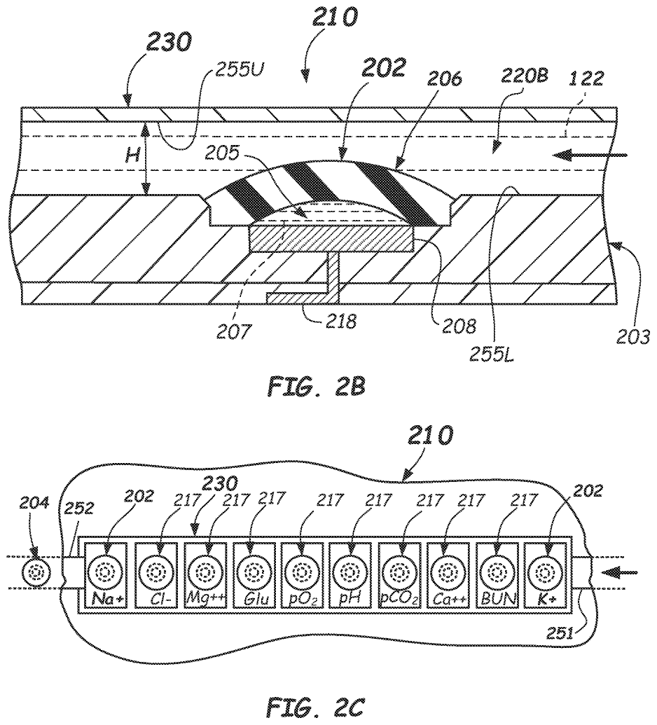

Referring again to FIG. 2A, a bottom view of a diagnostic cartridge 210 comprising one or more of the ion selective electrode sensors 202 is shown. A sample passageway 220 can extend from a sample inlet 209 (on the opposite side of the bottom side shown) into a cartridge body 211. In the depicted embodiment, the sample passageway 220 can comprise a first portion 220A extending from the sample inlet 209 to a second portion 220B. Second portion 220B can comprise a sensor array 230 therein made up of multiple sensors including one or more of the ion selective electrode sensors 202. Sensor array 230 may optionally include one or more additional sensors 217, a reference sensor, and possibly a ground. Optionally, a reference sensor 204 can be part of the diagnostic analyzer or may be located on a fluid pathway that is not in the sensor array 230, as shown in FIG. 2A. The second portion 220B can have different dimensions as compared to the first portion 220A. For example, the second portion 220B may be wider to accommodate, for example, the dimensions of the various sensors 202, 217, and/or ground housed in the sensor array 230. Thus, the second portion 220B may resemble a chamber in some non-limiting embodiments. Coupled at a downstream end of the second portion 220B can be a waste passageway 219 comprising a conduit or passage that is configured to receive the sample outflow from the sensor array 230. The waste passageway 219 can be located after the sample 122 contacts the last sensor or component in the sensor array 230, such as Na+ sensor 202 shown in FIGS. 2A and 2C. Optionally, reference sensor 204 may be located in the waste passageway 219 as shown in FIG. 2C, or elsewhere in the flow path.

Sample passageway 220 can have a cross-sectional area of from about 12,500 μm² to about 0.8 mm², for example. In some non-limiting embodiments, the sample passageway 220 can have a width-to-height ratio W:H that may be about 5:1 or greater. Height H the dimension across the sample passageway 220 as shown in FIG. 2B, whereas the width W across the sample passageway 220 and length L is as shown in FIG. 2A. Width W may be from about 250 μm to about 2 mm, and a height H may be from about 50 μm to about 400 μm. The length L along the sample passageway 220 from the sample inlet 209 to the start of the waste passageway 219 may be from about 1.25 mm to about 20 mm or greater. Other relationships between length L, width W, and/or height H may be employed and other suitable length L, height H, and/or width W dimensions may be used.

In more detail, the sensor array 230 of the sensor assembly 201, as shown, can comprise a first and second ion selective electrode sensors 202 configured to contact the sample 122 along the sample passageway 220. Both of the sensors 202 may be provided in the second portion 220B as shown in the depicted embodiments of FIGS. 2A and 2C. Other additional sensors 217 may be provided and located in the sensor array 230.

In more detail, and in further reference to FIG. 2B, the one or more ion selective electrode sensors 202 comprise an electrode body 203 having an ion selective membrane (ISM) 206 coupled thereto. The electrode body 203 can be formed of any suitable insulator, such as an insulating polymer (e.g., epoxy or the like). The electrode body 203 may be continuous along the length of the sensor array 230, such that the bodies of each of the respective sensors 202, 217 can be interconnected. The upper wall 255U and the sensors (e.g., ISM 202 and other sensors 217) and a lower wall 255L form the second portion 220B of sample passageway 220 and the second portion 220B receives the sample 122 therein. Flow direction is shown by way of example, but could be different as could the arrangement of the sensors.

The ISM 206 is formed as a thin polymer sheet that is selective to certain monovalent cations, such as Na+ and K+ monovalent cations as described herein. The ISM 206 may have a diameter of from about 1500 μm to about 1700 μm and a thickness of from about 10 μm to about 100 μm, for example. Other suitable diameters, dimensions, and/or thickness may be used. The chemical composition of the ISM 206 making it selective for Na+ or K+ cations can be any of the chemical compositions comprising from about 0.6 wt % to about 1.8 wt % boron-containing salt as are described herein in Tables 1 and 2 below.

Attachment of the ISM 206 to the electrode body 203 may be by any suitable means, such as bonding with an adhesive, compression and sealing using a sealing ring, or other suitable attachment means for sealing the membrane-body interface. Once attached, an internal reservoir 205 may be formed by the ISM 206 and the walls of the electrode body 203 and an electrode 208 in some non-limiting embodiments. Such as reservoir 205 can be filled with a suitable electrolyte solution 207. Electrolyte solution 207 can be any suitable salt solution, such as NaCl or KCl or a mixture of multi-electrolyte salt solution, for example. Optionally, the electrolyte solution 207 may be an electrolyte gel such as a hydrogel. The hydrogel may comprise a methocel material in some non-limiting embodiments. Other suitable electrolyte solutions may be used.

The ISM 206 may be formed from a semi-permeable material, such as a polymer material and may be provided in direct contact with the sample 122 as shown in FIG. 2B. For example, the polymer material may be an inert polytetrafluoroethylene (PTFE) material, a PVC material, a polyurethane material, or the like.

The ion selective electrode sensor 202 of FIG. 2B having a solid state integrated chip structure can be connected to an inlet 251 and an outlet 252 of a diagnostic analyzer (not shown) or otherwise such inlet 251 and outlet 252 can comprise the sample passageway 220 and waste passageway 219 of a diagnostic cartridge 210 as shown in FIG. 2C. The inlet 251 supplies the sample 122 to the sensor array 230 including the one or more ion selective sensors 202. The diagnostic cartridge 210 and sensor array 230 may include one or more other sensors 217 configured to measure one or more other analytes and/or conditions, such as Cl−, Mg++, Glu, pO₂, pH, pCO₂, Ca++, BUN, and the like. Other additional sensors 217 that are configured to sense other analytes and/or conditions such as Hct, Lac, and Crea may be included in addition or in substitution thereof. The sensor array 230 may further include a reference electrode configured for providing a reference signal or optionally the reference sensor 204 may be provide outside of the diagnostic cartridge 210 as shown in FIG. 2C in some non-limiting embodiments.

The one or more ion selective electrode sensors 202, the one or more reference electrodes 204, and possibly a ground can be electrically coupled to a detection system (not shown), which may include suitable electronics to enable providing a suitable bias and reading an electrical potential change (or current change) between the ion selective electrode sensor 102, the ground, and/or the reference electrode 204 as a measurable signal. Such signal processing is known to those of skill in the art and need not be further described herein. The reference sensor 204 and ground construction are well known and will not be described further herein. For example, the reference system, the reference sensor 204, and the ground can be of the type used in blood analysis system available from Siemens Medical Solutions.

Again referring to FIG. 2B, the ISM 206 further comprises an electrode 208 in contact with the electrolyte solution 207. Electrode 208 can be of any suitable construction, such as an electrically conductive trace that extends to an electrical contact 218 provided on the body of the diagnostic cartridge 210 (e.g., on a bottom thereof) that is interconnected to the diagnostic analyzer as the diagnostic cartridge 210 is coupled thereto, for example. For example, the electrode 208 can comprise a silver (AG) element which can be coated with a silver chloride (AGCl) coating, gold, platinum, combination of the aforementioned, or the like, for example. In other non-limiting embodiments, the electrode 208 may be made of other suitably electrically conductive materials. The connection between the electrode 208 and the electrical contact 218 provided on the body of the diagnostic cartridge 210 can be any suitable electrically conductive material, such as described above, and may be formed integrally or separately from the electrode 208, but in electrical contact therewith. As shown in FIG. 2A, the electrical contact 218, is shown for simplicity, as a single electrical contact 218. However, in the sensor array 230, there will be one electrical contact 218 for each sensor 202, 217, and ground, if present, that is connectable to a diagnostic analyzer (not shown) so that the signals produced by the sensors may be processed by the processor of the diagnostic analyzer.

According to one or more non-limiting embodiments herein, the chemical composition of the ion selective membrane 106, 206 comprises the following ingredients: a polymer such as polyvinyl chloride (PVC), a boron-containing salt in a weight percentage concentration of from about 0.6 wt % to about 1.8 wt %, based on the total weight of all non-solvents in the chemical composition, a plasticizer, and at least one monovalent selective ionophore (i.e., at least one Na+ or K+ selective ionophore). Each of these ingredients and the performance of the membranes 106, 206 are described in detail herein with reference to Table 1 and Table 2.

### Polymer Membrane Base Material

A polymer, such as Polyvinyl chloride (PVC) can be provided in an amount of from about 28 wt % to about 55 wt %, based on the total weight of 100% of the non-solvents in the chemical composition. In some non-limiting embodiments, the wt % of PVC can range from about 28 wt % to about 40 wt %, or even from about 28 wt % to about 35 wt %, based on the total weight of all non-solvents in the composition. The poly(vinyl chloride) (PVC) used in the membrane composition can be any suitable relatively high molecular weight PVC (e.g., PVC with a molecular weight of ≥418 g/mol). For example, product 81392 (Selectophore Grade PVC) available from Sigma-Aldrich, Inc. PVC can be used. Optionally, PTFE or a polyurethane could be used.

### Boron-Containing Salt

The boron-containing compound in the membrane composition comprises a boron-containing salt. For example, a lipophilic boron-containing salt (e.g., lipophilic borate) can be used. In some non-limiting embodiments, the boron-containing salt (e.g., lipophilic borate) can comprise potassium tetrakis (4-chlorophenyl) borate (KTpClPB), for example, with an empirical formula of (ClC₆H₄)₄BK. Optionally, the boron-containing salt can be sodium tetrakis[3,5-bis(trifluoromethyl)phenyl]borate (NaTFPB) with an empirical formula C₃₂H₁₂BF₂₄Na.

The boron-containing salt (e.g., lipophilic borate) is provided in a weight percentage of from about 0.6 wt % to about 1.8 wt %, based on the total weight of all non-solvents in the chemical composition. In certain non-limiting embodiments, the boron-containing salt can be provided in from about 0.7 wt % to about 1.7 wt %, and in other non-limiting embodiments from about 0.9 wt % to about 1.6 wt %, all based on the total weight of all non-solvents in the chemical composition. The higher wt % in the range (e.g., range of from about 0.9 wt % to about 1.6 wt %) can have the advantage of neutralizing the benzalkonium cations at the phase boundary so that the membrane response signal substantially corresponds to the charge-separation potential signal from Na+/ionophore chelation reaction with minimal or no BK interference. However, if the boron-containing salt wt % is too high, such as provided at about 2.0 wt % or above, excessive borate content in the ISM competes with Na+/ionophore chelation at the phase boundary between the PVC and the sample 122 and even substantially dominates phase boundary potential signal (charge separation potential), which follows ion-exchange Hofmeister order. Therefore, ionophore selectivity for Na+ against other cations (e.g. K+, Ca2+, etc.) is deteriorated and sensor response selectivity to Na+ can be substantially lost, i.e., it has severe impact.

### Ionophores

The chemical composition of the ISM membrane further comprises at least one ionophore. Ionophores are compounds that form complexes with specific ions and therefore facilitate their transport across the polymer membrane. An ionophore typically has a hydrophilic pocket (or hole) that forms a binding site specific for a particular ion. The ion selective membranes herein comprise chemical compositions that are selective for particular monovalent cations. For example, compositions selective for sodium (Na+) monovalent cations include Na+ ionophores. Likewise, compositions selective for potassium (K+) monovalent cations include K+ ionophores. One or more ionophores can be included in an amount of greater than or equal to about 2.0 wt %, or even in a range of from about 2.0 wt % to about 11.0 wt %, based on the total weight of all non-solvents in the chemical composition. In other non-limiting embodiments, the ionophore(s) can be included in an amount of greater than or equal to about 3.0 wt %, or even in a range of from about 3.0 wt % to about 11.0 wt %, based on the total weight of all non-solvents in the chemical composition.

Where the membrane 106, 206 is specifically selective for monovalent Na+ cations, the membrane composition can contain at least one sodium (Na+) monovalent selective ionophore. Non-limiting examples of sodium (Na+) monovalent selective ionophores that can be utilized in accordance with the present disclosure include N,N,N′,N′-Tetracyclohexyl-1,2-phenylenedioxydiacetamide (Empirical formula C₃₄H₅₂N₂O₄ and available as ETH2120 from Sigma-Aldrich, Inc.), and/or optionally 4-tert-Butylcalix[4]arene-tetraacetic acid tetraethyl ester (Empirical formula C₆₀H₈₀O₁₂ and available as Sodium Ionophore X from Sigma-Aldrich, Inc.). When one or more Na+ selective ionophores is provided in the chemical composition, the Na+ ionophore(s) can be included in an amount of greater than or equal to about 3.0 wt %, or even from about 3.0 wt % to about 11.0 wt %, based on the total weight of all non-solvents in the chemical composition.

A molar ratio of boron-containing salt to Na+ ionophore (B/I ratio) can be in the range from about 0.04 to about 0.7, or even about 0.04 to about 0.6 in some non-limiting embodiments. A molar ratio of boron-containing salt to K+ ionophore (B/I ratio) can be in the range from about 0.3 to about 0.9, or even about 0.5 to about 0.8 in some non-limiting embodiments, with the weight percentage of K+ ionophore being greater than or equal to about 2.0 wt %, and may range from about 2.0 wt % to about 5.0 wt %, or even about 2.0 wt % to about 4.0 wt % (based on the total weights of non-solvents in the chemical composition).

Where the membrane 106, 206 is specifically selective for K+ monovalent cations, the membrane composition can contain at least one potassium (K+) monovalent selective ionophore. Non-limiting examples of potassium (K+) ionophores that can be utilized in accordance with the present disclosure include Valinomycin (Empirical formula C₅₄H₉₀N₆O₁₈) and/or optionally 2-Dodecyl-2-methyl-1,3-propanediyl bis[N-[5′-nitro (benzo-15-crown-5)-4′-yl]carbamate] (Empirical formulation C₄₆H₇₀N₄O₁₈ and available as BME44 with from Sigma-Aldrich, Inc.).

As is shown in FIG. 2C, in some blood gas analyzers, a diagnostic cartridge 210 can be used to house one or more of the ISE sensors (e.g., ISE sensors 202). In particular, some point-of-care blood gas analyzers can include 5 or more different ISE sensors within a single diagnostic cartridge 210, wherein the cartridge can be inserted into, and detached from, the diagnostic analyzer and wherein each ISE sensor can be used multiple times in some non-limiting embodiments. The diagnostic cartridge 210 or the analyzer itself can include therein control systems enabling calibration and/or wash systems enabling washing to allow multiple uses (i.e., multiple tests by resusing the diagnostic cartridge 210). Among the available ISE sensors, diagnostic cartridges 210 containing Na+ and K+ ISE sensors 102, 202 can exhibit benzalkonium (BK) interference. Other targets, such as pH, pCO₂, and Cl− do not have a BK interference issue as they are based on different chemical sensing mechanism. Further, multivalent cations (e.g., Ca++ and Mg++) also experience little BK interference. Therefore, the present disclosure is directed predominantly at Na+ and K+ ISE sensors 102, 202 that can exhibit substantial benzalkonium interference.

### Plasticizer

The plasticizer used in the ion selective membrane composition can be any suitable plasticizer. Monovalent ISMs 106, 206 (e.g., specifically selective for Na+ and K+ cations) can use a plasticizer with low dielectric constant (e.g., epsilon ≤5.0, or even ≤4.0) and high lipophilicity (e.g., log P≥11). Sebacate, such as dioctyl sebacate (DOS), can be used as the plasticizer when the at least one ionophore comprises at least one Na+ ionophore or at least one K+ ionophore. DOS is an organic compound and, in particular, an ester of sebacic acid and 2-Ethylhexanol. Phthalate can also be used as a plasticizer in such monovalent ISE membranes in cases where its toxicity is not a concern. The plasticizer can be provided in from about 45 wt % to about 70 wt %, based on the total weight of all non-solvents in the chemical composition. When dioctyl sebacate is the plasticizer, it can be provided at a concentration in a range of from about 45 wt % to about 70 wt %, or even a range of from about 55 wt % to about 65 wt %, based on the total weight of all non-solvents in the chemical composition.

### Solvent

The solvent used to disperse the non-solvents in the membrane solution can be any suitable solvent, such as a heterocyclic compound (e.g., a cyclic ether). One example solvent that can be used with Na+ and K+ monovalent ion selective membrane compositions can be Tetrahydrofuran (THF), otherwise referred to as oxolane. THF is an organic compound with the formula (CH2)4O that has a relatively low boiling point and can advantagesly dissolve a wide variety of organic compounds. It is a colorless, water-miscible organic liquid with suitably low viscosity. It can be used as a precursor to the formation of the ISM 106, 206.

Another example solvent that can be used with Na+ and K+ ion selective membrane compositions can be cyclohexanone (otherwise known as pimelic ketone, oxocyclohexane, cyclohexyl ketone, and referred to as “CYC” herein). CYC is an organic compound that has a clear oily liquid appearance, but may have light yellow tinge and has an odor reminiscent of acetone. CYK belongs to the class of cyclic ketones (organic compounds) with the formula (CH₂)₅CO, and is thus a six carbon cyclic molecule. In some non-limiting embodiments, both THF and CYC can be used in combination. In this case, the ratio of THF:CYC can be from about 1.0:0.0 to about 0.0:1.0, for example. In some non-limiting embodiments, a ratio of about 1:0.25 (THF:Cyclohexanone) can be used for excellent solvent evaporation.

The solvent can be provided in a wt % of from about 70 wt % to about 92 wt %, or even from about 85 wt % to about 92 wt %, based upon the total weight percent of solvents and non-solvents in the membrane solution. In some non-limiting embodiments, heating may be involved in the evaporation process to aid in forming the chemical composition into an ISM 106, 206. Heating temperature ranges for evaporation can be from about 20 °C to about 30 °C, for example. However, slowing down the evaporation speed can help to produce a substantially more homogeneous polymeric ISM 106, 206.

### Examples

Example weights (in mg) and weight percentages (wt %) for the boron-containing salt, ionophore, and the mole ratio for boron-containing salt to ionoiphore (B/I ratio), for Na+ and K+ monovalent ISM 106, 206 are listed in Tables 1 and 2 below. Example wt % for the chemical compositions for Na+ and K+ monovalent ion selective membranes (ISM) 106, 206 are listed below in Table 2. The chemical composition of matter of the polymeric ion selective membranes 106, 206, comprises: a polymer such as PVC, a boron-containing salt provided in a weight percentage of from about 0.6 wt % to about 1.8 wt %, based on the total weight of all the non-solvents in the chemical composition, a plasticizer, and the monovalent selective ionophore(s). The membrane solution used to form the membranes 106, 206 includes the above plus a suitable solvent (e.g., combination of CYC and THF). The boron-containing salt is referred to in Table 1 as borate. The impact level of induced offset bias is given by the Impact Level Key listed below.

TABLE 1
Weights for Na+ and K+ membrane examples

| | | | | Boron- | | | Total non- | |
|---|---|---|---|---|---|---|---|---|
| Selective | | | | containing | | | solvent | |
| Sensor | | Ionophore | Ionophore | salt | PVC | Plasticizer | content | Solvent |
| type | # | Type | (mg) | (mg) | (mg) | (mg) | (mg) | (mg) |
| Na+ | C | ETH2120 | 8.0 | 3.2 | 62.0 | 126 | 199.2 | 2000 |
| | D | ETH2120 | 10.0 | 0.8 | 62.0 | 127 | 199.8 | 2000 |
| | F | ETH2120 | 20.0 | 1.6 | 59.0 | 120 | 200.6 | 2000 |
| | I | Na-X | 10.0 | 2.4 | 62.0 | 126 | 200.4 | 2000 |
| | J | Na-X | 10.0 | 10.0 | 59.4 | 121 | 200.4 | 2000 |
| K+ | M | Valinomycin | 6.0 | 1.4 | 64.0 | 129 | 200.4 | 2000 |
| | O | Valinomycin | 6.0 | 2.0 | 63.0 | 129 | 200.0 | 2000 |

TABLE 2
Wt % for Na+ and K+ composition examples

| | | | Boron- | B/I | | | | |
|---|---|---|---|---|---|---|---|---|
| Sensor | | Ionophore | containing | Molar | PVC | Plasticizer | Solvent | |
| type | # | wt % | salt wt % | Ratio | wt % | wt % | wt % | Impact |
| Na+ | C | 4.0% | 1.6% | 0.50 | 31.1% | 63.3% | 90.9% | Minimal |
| | D | 5.0% | 0.4% | 0.04 | 31.0% | 63.6% | 90.9% | Large |
| | F | 10.0% | 0.8% | 0.04 | 29.4% | 59.8% | 90.9% | Medium |
| | I | 5.0% | 1.2% | 0.50 | 30.9% | 62.9% | 90.9% | Minimal |
| | J | 5.0% | 5.0% | 1.00 | 29.6% | 60.4% | 90.9% | Severe |
| K+ | M | 3.0% | 0.7% | 0.55 | 31.9% | 64.4% | 90.9% | Medium |
| | O | 3.0% | 1.0% | 0.70 | 31.5% | 64.5% | 90.9% | Minimal |

Impact Level Key:
Minimal: <10% induced bias % by 25 ug/mL BK
Medium: 10% to 50% induced bias % by 25 ug/mL BK
Large: >50% to 100% induced bias % by 25 ug/mL BK
Severe: >100% Severe-response sensitivity is lost

Table 2 illustrates various chemical composition examples at various wt % of the boron-containg salt (e.g., KTpClPB) and wt % of Na+ and K+ ionophores that are desired in order to manufacture ISMs 106, 206 that effectively minimize BK interference in PVC-based ISE sensors 102, 202. As can be seen from Table 2, any sensing membrane containing high borate wt % (>2.0 wt % such as in severe example J), such Na+ and K+ selective sensors will substantially lose their Na+ or K+ cation response sensitivity (slope is reduced by 80% of Nernstian response).

Also shown in Table 2 is that when the boron-containg salt wt % (borate wt %) is greater than or equal to about 0.6 wt % and less than or equal to about 1.8 wt %, then minimal or medium impact level due to BK exposure is provided to the ISE sensor 102, 202. Table 2 illustrates chemical compositions for examples of membranes 106, 206 (examples C, I, and O) that exhibit minimal induced bias offset %, i.e., less than about 10% induced bias % when exposed to about 25 μg/mL BK.

Table 2 above also illustrates that a B/I molar ratio of moles of the boron-containing salt (B) to moles of ionophore(s) (I) can be greater than or equal to about 0.04 for wt % ionophores of greater than about 2.0 wt % (based on the total weight of non-solvents in the chemical composition), for both polymeric Na+ and K+ ISMs 106, 206. Increasing borate wt % in PVC to the range of from about 0.6 wt % to about 1.8 wt %, or even a range of from about 0.9 wt % to about 1.6 wt %, can significantly reduce or even substantially eliminate BK interference for Na+ and K+ quantification in blood gas assay testing. With this ISM membrane composition for ISE sensors 102, 202, BK interference can be reduced and thus blood gas analyzer results can be more reliable. Other example compositions that have large BK interference are shown below in Table 3.

TABLE 3
Further Membrane Examples with Large BK Impact

| | | | Borate | | |
|---|---|---|---|---|---|
| | | | (KTpCIPB) | | |
| | | Ionophore | to Ionophore | Borate | Impact |
| Ionophore | Membrane | wt % | (mol %) | wt % | level |
| ETH 2120 | A | 1% | 0% | 0.1% | large |
| for Na+ | B | 1% | 50% | 0.5% | large |
| Na-X | G | 1% | 0% | 0.0% | large |
| for Na+ | H | 1% | 50% | 0.2% | large |
| | K | 10% | 0% | 0.0% | large |
| Valinomycin | L | 1% | 20% | 0.2% | large |
| For K+ | N | 3% | 90% | 2.2% | large |

### Manufacturing Method

The ISM 106, 206 can be formed by the following manufacturing method. First the membrane solution is prepared by weighing and mixing the non-solvent components according to composition formulations described herein. The components are placed in a suitable vessel (e.g., a 10 ml glass vial) one after another: boron-containing salt, ionophore, polymer (e.g., PVC), then plasticizer. An appropriate volume of organic solvent (e.g., THF or THF/CYC combination) is added to dissolve the non-solvent components and stirred until clear. For example, a magnetic stir bar in the vessel placed on a stir plate can be used for a suitable time (e.g., about 12 hours) at room temperature until a clear ISE membrane solution is obtained.

The manufacturing method can utilize a dispensing and evaporation method to form the ISM 106, 206. The weight % of non-solvent components to weight % of the solvent can be from about 8 wt % to about 30 wt % (non-solvent components) to about 70 wt % to about 92 wt % (solvent components), respectively. In particular, a 9 wt % non-solvent/90 wt % solvent ratio can be used for Na+ and K+. Membrane solutions can be stored in refrigerator, such as at about 4 °C.

Formation of the Ion selective membrane 106, 206 can include providing a formation vessel, such as a glass ring (e.g., approx. 20 mm radius) laid on a glass plate, for example. Other suitable containers can be used. Approximately 2 mL of the ion selective membrane solution is deposited into the glass ring, and the membrane solution is left in the glass ring a sufficient amount of time for solvent evaporation, thus obtaining a thin PVC membrane precursor (e.g., thickness of approx. 50 μm-200 μm) that is formulated to be selected for a particular cation (e.g., Na+ or K+). Evaporation can be accomplighed at room temperature or through supplying supplemental heating.

To form the ion selective membrane 106, 206, a small disc (e.g., 6 mm diameter for FIG. 1 and 1500 μm to 1700 μm for FIG. 2B embodiments) can be excised from the cast PVC membrane precursor and it can be applied to the electrode body 103, 203 (e.g., a Philips electrode body 103 as shown in FIG. 1 or an electrode body 203 of the integrated sensor 202 as shown in FIG. 2B).

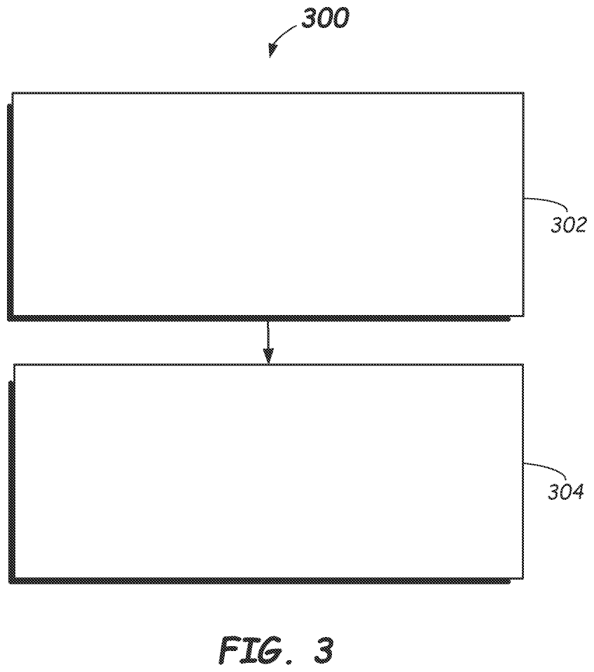

FIG. 3 illustrates a flowchart depicting a method 300 of minimizing sensor interference in an ion selective electrode sensor 102, 202. The method 300 comprises, in block 302, providing an ion selective electrode sensor (e.g., ion selective electrode sensor 102, 202) comprising an ion selective membrane (e.g., ion selective membrane 106, 206), the ion selective membrane having a chemical composition further comprising: a polymer (e.g., polyvinyl chloride (PVC)), a boron-containing salt, a plasticizer, and at least one monovalent selective ionophore. The at least one monovalent selective ionophore can be selective for Na+ or K+ as described herein. Boron-containing salt can be provided in a weight percentage of from about 0.6 wt % to about 1.8 wt %, based on the total weight of all non-solvents in the chemical composition. Example chemical compositions of membranes 106, 206 are described in Tables 1 and 2 herein.

The method 300 further comprises, in block 304, exposing the ion selective membrane to a Benzalkonium-containing cleaner. The exposing may be a consequence of periodic cleaning of the blood gas diagnostic analyzer to remove blood, debris, or other contamination therefrom. According to the method 300, in block 304, upon exposing the ion selective membrane, the ion selective sensor 102, 202 has less than about 10.0% induced bias offset, less than about 5.0% induced bias offset, less than about 4.0% induced bias offset, or even less than about 2.0% induced bias offset in some non-limiting embodiments, all upon exposure of the ion selective membrane (e.g., ion selective membrane 106) to the Benzalkonium-containing cleaner having a concentration of benzalkonium of about 25 μg/mL. In other non-limiting embodiments, the ion selective sensor 102, 202 has less than or equal to about +1.6% induced bias offset upon exposure of the ion selective membrane (e.g., ion selective membrane 106) to the Benzalkonium-containing cleaner having a concentration of benzalkonium of about 25 μg/mL. A measurement without BK is the baseline using normal blood (having 130-150 mmol/L Na) as compared to the same blood with concentration of benzalkonium of about 25 μg/mL in order to obtain the induced bias offset as a percentage, as follows:

Induced Bias Offset (%)={(Measurement with BK exposure−Measurement without BK exposure)/Measurement without BK exposure}*100.

For example, the measurement without BK exposure can be taken prior to BK (25 μg/mL) exposure and can have a value of Na+=135 mM. The measurement with BK exposure can be Na+=162 mM. Accordingly, then the Induced Bias Offset (%)=(162 mM−135 mM)/135 mM)*100=20%.

### Non-Limiting Illustrative Embodiments

The following is a list of non-limiting illustrative embodiments disclosed herein:

Illustrative embodiment 1. A polymeric ion selective electrode sensor, comprising: a polymeric membrane selective for monovalent cations having a chemical composition comprising a boron-containing salt at a concentration in a range of from about 0.6 wt % to about 1.8 wt %, based on a total weight of all non-solvents in the chemical composition.

Illustrative embodiment 2. The polymeric ion selective electrode sensor of illustrative embodiment 1, wherein the chemical composition further comprises at least one ionophore.

Illustrative embodiment 2A. The polymeric ion selective electrode sensor of illustrative embodiment 2, wherein the at least one ionophore comprises at least one monovalent cation selective ionophore.

Illustrative embodiment 3. The polymeric ion selective electrode sensor of any one of the preceding illustrative embodiments, wherein a molar ratio of the boron-containing salt to the at least one ionophore is ≥about 0.04.

Illustrative embodiment 4. The polymeric ion selective electrode sensor of any one of the preceding illustrative embodiments, wherein a molar ratio of the boron-containing salt to the at least one ionophore is in a range of from about 0.04 to about 0.9.

Illustrative embodiment 5. The polymeric ion selective electrode sensor of any one of the preceding illustrative embodiments, wherein the molar ratio of the boron-containing salt to the at least one ionophore is in a range of from about 0.3 to about 0.8.

Illustrative embodiment 6. The polymeric ion selective electrode sensor of any one of the preceding illustrative embodiments, wherein the chemical composition of the polymeric membrane comprises at least one Na+ ionophore.

Illustrative embodiment 7. The polymeric ion selective electrode sensor of any one of the preceding illustrative embodiments, wherein a molar ratio of the boron-containing salt to the at least one Na+ ionophore is in a range of from about 0.04 to about 0.7.

Illustrative embodiment 8. The polymeric ion selective electrode sensor of any one of the preceding illustrative embodiments, wherein the at least one Na+ ionophore comprises N, N, N′,N′-Tetracyclohexyl-1,2-phenylenedioxydiacetamide and/or 4-tert-Butylcalix[4]arene-tetraacetic acid tetraethyl ester.

Illustrative embodiment 9. The polymeric ion selective electrode sensor of any one of the preceding illustrative embodiments, wherein the at least one Na+ ionophore is provided at a concentration in a range of from about 2.0 wt % to about 7.0 wt %, based on the total weight of all non-solvents in the chemical composition.

Illustrative embodiment 10. The polymeric ion selective electrode sensor of any one of the preceding illustrative embodiments, wherein the chemical composition of the polymeric membrane comprises at least one K+ ionophore.

Illustrative embodiment 11. The polymeric ion selective electrode sensor of any one of the preceding illustrative embodiments, wherein the at least one K+ ionophore comprises Valinomycin.

Illustrative embodiment 12. The polymeric ion selective electrode sensor of any one of the preceding illustrative embodiments, wherein the at least one K+ ionophore is provided at a concentration in a range of from about 2.0 wt % to about 5.0 wt %, based on the total weight of all non-solvents in the chemical composition.

Illustrative embodiment 13. The polymeric ion selective electrode sensor of any one of the preceding illustrative embodiments, wherein a molar ratio of the boron-containing salt to the at least one K+ ionophore is in a range of from about 0.3 to about 0.9.

Illustrative embodiment 14. The polymeric ion selective electrode sensor of any one of the preceding illustrative embodiments, wherein the chemical composition of the polymeric membrane further comprises at least one plasticizer.

Illustrative embodiment 15. The polymeric ion selective electrode sensor of any one of the preceding illustrative embodiments, wherein the at least one plasticizer is provided at a concentration in a range of from about 45 wt % to about 70 wt %, based on the total weight of all non-solvents in the chemical composition.

Illustrative embodiment 15A. The polymeric ion selective electrode sensor of any one of the preceding illustrative embodiments, wherein the at least one plasticizer comprises dioctyl sebacate.

Illustrative embodiment 16. The polymeric ion selective electrode sensor of any one of the preceding illustrative embodiments, wherein the chemical composition of the polymeric membrane further comprises polyvinyl chloride.

Illustrative embodiment 17. The polymeric ion selective electrode sensor of any one of the preceding illustrative embodiments, wherein the polyvinyl chloride is provided at a concentration in a range of from about 28 wt % to about 55 wt %, based on the total weight of all non-solvents in the chemical composition.

Illustrative embodiment 18. The polymeric ion selective electrode sensor of any one of the preceding illustrative embodiments, wherein the boron-containing salt is provided at a concentration in a range of from about 0.9 wt % to about 1.6 wt %, based on the total weight of all non-solvents in the chemical composition.

Illustrative embodiment 19. The polymeric ion selective electrode sensor of any one of the preceding illustrative embodiments, wherein the boron-containing salt comprises lipophilic borate.

Illustrative embodiment 20. The polymeric ion selective electrode sensor of any one of the preceding illustrative embodiments, wherein the lipophilic borate comprises potassium tetrakis (4-chlorophenyl) borate and/or sodium tetrakis[3,5-bis(trifluoromethyl)phenyl]borate.

Illustrative embodiment 21. The polymeric ion selective electrode sensor of any one of the preceding illustrative embodiments, further comprising at least one of: an electrode body having a sidewall, wherein the polymeric member is attached to a lower end of the electrode body, and wherein the sidewall of the electrode body and the polymeric member form a reservoir; an electrolyte solution disposed within the reservoir; and/or an electrode in contact with the electrolyte solution.

Illustrative embodiment 22. A diagnostic cartridge comprising a sensor array, wherein at least one sensor in the sensor array comprises the polymeric ion selective electrode sensors of any one of the preceding illustrative embodiments.

Illustrative embodiment 23. The diagnostic cartridge of any of the preceding illustrative embodiments, wherein the sensor array further comprises at least one multivalent cation electrode sensor.

Illustrative embodiment 24. The diagnostic cartridge of any of the preceding illustrative embodiments, wherein the sensor array further comprises at least one sensor that measures an analyte selected from the group consisting of Cl−, Mg++, Ca++, Glu, pO2, pH, pCO2, Ca++, BUN, Hct, Lac, and Crea.

Illustrative embodiment 25. The diagnostic cartridge of any of the preceding illustrative embodiments, wherein the sensor array further comprises at least one reference sensor.

Illustrative embodiment 26. A polymeric ion selective membrane for a polymeric monovalent cation selective electrode sensor, the membrane comprising: a polymer; a boron-containing salt provided at a concentration in a range of from about 0.6 wt % to about 1.8 wt %, based on a total weight of all non-solvents in the chemical composition of the membrane; a plasticizer; and at least one monovalent selective ionophore.

Illustrative embodiment 27. The polymeric ion selective membrane of any one of the preceding illustrative embodiments, wherein the boron-containing salt comprises potassium tetrakis (4-chlorophenyl) borate and/or sodium tetrakis[3,5-bis(trifluoromethyl)phenyl]borate.

Illustrative embodiment 28. The polymeric ion selective membrane of any one of the preceding illustrative embodiments, wherein the at least one monovalent selective ionophore is further defined as comprising at least one Na+ ionophore.

Illustrative embodiment 29. The polymeric ion selective membrane of any one of the preceding illustrative embodiments, wherein the at least one Na+ ionophore comprises N,N,N′,N′-Tetracyclohexyl-1,2-phenylenedioxydiacetamide and/or 4-tert-Butylcalix[4]arene-tetraacetic acid tetraethyl ester.

Illustrative embodiment 30. The polymeric ion selective membrane of any one of the preceding illustrative embodiments, wherein the at least one Na+ ionophore is provided at a concentration of greater than or equal to about 2.0 wt %, based on the total weight of all non-solvents in the chemical composition of the membrane.

Illustrative embodiment 31. The polymeric ion selective membrane of any one of the preceding illustrative embodiments, wherein the at least one Na+ ionophore is provided at a concentration in a range of from about 2.0 wt % to about 7.0 wt %, based on the total weight of all non-solvents in the chemical composition of the membrane.

Illustrative embodiment 32. The polymeric ion selective membrane of any one of the preceding illustrative embodiments, wherein the at least one monovalent selective ionophore is further defined as comprising at least one K+ ionophore.

Illustrative embodiment 33. The polymeric ion selective membrane of any one of the preceding illustrative embodiments, wherein the at least one K+ ionophore comprises Valinomycin and/or 2-Dodecyl-2-methyl-1,3-propanediyl bis[N-[5′-nitro (benzo-15-crown-5)-4′-yl]carbamate].

Illustrative embodiment 34. The polymeric ion selective membrane of any one of the preceding illustrative embodiments, wherein the plasticizer is dioctyl sebacate when the at least one monovalent selective ionophore is at least one K+ ionophore or at least one Na+ ionophore.

Illustrative embodiment 35. A method of minimizing sensor interference in an ion selective electrode sensor, the method comprising the steps of: providing an ion selective electrode sensor of any one of the preceding illustrative embodiments; and exposing the ion selective membrane to a cleaning agent comprising benzalkonium; and wherein the ion selective electrode sensor has less than +10.0% induced bias offset upon exposure to the benzalkonium-containing cleaning agent.

Illustrative embodiment 36. The method of minimizing sensor interference of any one of the preceding illustrative embodiments, wherein the ion selective electrode sensor comprises an ion selective membrane, and wherein the ion selective membrane has a chemical composition comprising: a polymer, a boron-containing salt, a plasticizer, and at least one monovalent selective ionophore

Illustrative embodiment 37. The method of minimizing sensor interference of any one of the preceding illustrative embodiments, wherein the cleaning agent has a benzalkonium concentration of at least about 25 μg/mL.

Illustrative embodiment 38. The method of minimizing sensor interference of any one of the preceding illustrative embodiments, wherein the ion selective electrode sensor has less than +5.0% indiced bias offset upon exposure to the benzalkonium-containing cleaning agent.

Illustrative embodiment 39. The method of minimizing sensor interference of any one of the preceding illustrative embodiments, wherein the boron-containing salt is provided at a concentration in a range of from about 0.6 wt % to about 1.8 wt %, based on a total weight of all non-solvents in the chemical composition of the membrane.

Illustrative embodiment 40. A method of minimizing sensor interference in an ion selective electrode sensor, the method comprising the steps of: measuring a concentration of at least one monovalent cation in a first sample using an ion selective electrode sensor of any one of the preceding illustrative embodiments; exposing the ion selective membrane to a cleaning agent comprising benzalkonium; and measuring a concentration of at least one monovalent cation in a first sample using an ion selective electrode sensor of any one of the preceding illustrative embodiments; wherein the ion selective electrode sensor has less than +10.0% induced bias offset upon exposure to the benzalkonium-containing cleaning agent.

Illustrative embodiment 41. The method of minimizing sensor interference of any one of the preceding illustrative embodiments, wherein the ion selective electrode sensor comprises an ion selective membrane, and wherein the ion selective membrane has a chemical composition comprising: a polymer, a boron-containing salt, a plasticizer, and at least one monovalent selective ionophore

Illustrative embodiment 42. The method of minimizing sensor interference of any one of the preceding illustrative embodiments, wherein the cleaning agent has a benzalkonium concentration of at least about 25 μg/mL.

Illustrative embodiment 43. The method of minimizing sensor interference of any one of the preceding illustrative embodiments, wherein the ion selective electrode sensor has less than +5.0% indiced bias offset upon exposure to the benzalkonium-containing cleaning agent.

Illustrative embodiment 44. The method of minimizing sensor interference of any one of the preceding illustrative embodiments, wherein the boron-containing salt is provided at a concentration in a range of from about 0.6 wt % to about 1.8 wt %, based on a total weight of all non-solvents in the chemical composition of the membrane.

Although embodiments are described herein with reference to specific examples, the scope of the disclosure is not intended to be limited to the details and specific examples described herein. Rather, various modifications may be made to the embodiments and details within the scope and range of equivalents of the claims.

What is claimed is:

1. A polymeric ion selective electrode sensor, comprising:
a polymeric membrane selective for monovalent cations, the polymeric membrane comprising:
a boron-containing salt at a concentration in a range of from about 0.6 wt % to about 1.8 wt %, based on a total weight of all non-solvents in the membrane; and
at least one monovalent cation selective ionophore.

2. The polymeric ion selective electrode sensor of claim 1, wherein a molar ratio of the boron-containing salt to the at least one ionophore is about 0.04 or higher.

3. The polymeric ion selective electrode sensor of claim 2, wherein a molar ratio of the boron-containing salt to the at least one ionophore is in a range of from about 0.04 to about 0.9.

4. The polymeric ion selective electrode sensor of claim 3, wherein the molar ratio of the boron-containing salt to the at least one ionophore is in a range of from about 0.3 to about 0.8.

5. The polymeric ion selective electrode sensor of claim 1, wherein the at least one ionophore comprises at least one Na+ ionophore.

6. The polymeric ion selective electrode sensor of claim 5, wherein a molar ratio of the boron-containing salt to the at least one Na+ ionophore is in a range of from about 0.04 to about 0.7.

7. The polymeric ion selective electrode sensor of claim 5, wherein the at least one Na+ ionophore comprises N, N, N′,N′-Tetracyclohexyl-1,2-phenylenedioxydiacetamide and/or 4-tert-Butylcalix[4]arene-tetraacetic acid tetraethyl ester.

8. The polymeric ion selective electrode sensor of claim 5, wherein the at least one Na+ ionophore is provided at a concentration in a range of from about 2.0 wt % to about 7.0 wt %, based on the total weight of all non-solvents in the polymeric membrane.

9. The polymeric ion selective electrode sensor of claim 1, wherein the at least one ionophore comprises at least one K+ ionophore.

10. The polymeric ion selective electrode sensor of claim 9, wherein the at least one K+ ionophore comprises Valinomycin.

11. The polymeric ion selective electrode sensor of claim 9, wherein the at least one K+ ionophore is provided at a concentration in a range of from about 2.0 wt % to about 5.0 wt %, based on the total weight of all non-solvents in the polymeric membrane.

12. The polymeric ion selective electrode sensor of claim 9, wherein a molar ratio of the boron-containing salt to the at least one K+ ionophore is in a range of from about 0.3 to about 0.9.

13. The polymeric ion selective electrode sensor of claim 1, wherein the polymeric membrane comprises dioctyl sebacate as a plasticizer.

14. The polymeric ion selective electrode sensor of claim 1, wherein a plasticizer is provided at a concentration in a range of from about 45 wt % to about 70 wt %, based on the total weight of all non-solvents in the polymeric membrane.

15. The polymeric ion selective electrode sensor of claim 1, wherein the polymeric membrane comprises polyvinyl chloride.

16. The polymeric ion selective electrode sensor of claim 15, wherein the polyvinyl chloride is provided at a concentration in a range of from about 28 wt % to about 55 wt %, based on the total weight of all non-solvents in the polymeric membrane.

17. The polymeric ion selective electrode sensor of claim 1, wherein the boron-containing salt is provided at a concentration in a range of from about 0.9 wt % to about 1.6 wt %, based on the total weight of all non-solvents in the polymeric membrane.

18. The polymeric ion selective electrode sensor of claim 1, wherein the boron-containing salt is lipophilic borate.

19. The polymeric ion selective electrode sensor of claim 18, wherein the lipophilic borate comprises potassium tetrakis (4-chlorophenyl) borate and/or sodium tetrakis[3,5-bis(trifluoromethyl)phenyl]borate.

20. The polymeric ion selective electrode sensor of claim 1, further comprising:
an electrode body having a sidewall, wherein the polymeric member is attached to a lower end of the electrode body, and wherein the sidewall of the electrode body and the polymeric member form a reservoir;
an electrolyte solution disposed within the reservoir; and
an electrode in contact with the electrolyte solution.

21. A diagnostic cartridge, comprising a sensor array, wherein at least one sensor in the sensor array comprises the polymeric ion selective electrode sensors of claim 1.

22. The diagnostic cartridge of claim 21, wherein the sensor array further comprises at least one multivalent cation electrode sensor.

23. The diagnostic cartridge of claim 21, wherein the sensor array further comprises at least one sensor that measures an analyte selected from the group consisting of Cl−, Mg++, Ca++, Glu, pO₂, pH, pCO₂, Ca++, BUN, Hct, Lac, and Crea.

24. The diagnostic cartridge of claim 21, wherein the sensor array further comprises at least one reference sensor.

25. A polymeric ion selective membrane for a polymeric monovalent cation selective electrode sensor, the polymeric membrane comprising:
a polymer;
a boron-containing salt provided at a concentration in a range of from about 0.6 wt % to about 1.8 wt %, based on a total weight of all non-solvents in the polymeric membrane;
a plasticizer; and
at least one monovalent selective ionophore.

26. The polymeric ion selective membrane of claim 25, wherein the boron-containing salt comprises potassium tetrakis (4-chlorophenyl) borate and/or sodium tetrakis[3,5-bis(trifluoromethyl)phenyl]borate.

27. The polymeric ion selective membrane of claim 25, wherein the at least one monovalent selective ionophore comprises at least one Na+ ionophore.

28. The polymeric ion selective membrane of claim 27, wherein the at least one Na+ ionophore comprises N,N,N′,N′-Tetracyclohexyl-1,2-phenylenedioxydiacetamide and/or 4-tert-Butylcalix[4]arene-tetraacetic acid tetraethyl ester.

29. The polymeric ion selective membrane of claim 27, wherein the at least one Na+ ionophore is provided at a concentration greater than or equal to about 2.0 wt %, based on the total weight of all non-solvents in the polymeric membrane.

30. The polymeric ion selective membrane of claim 27, wherein the at least one Na+ ionophore is provided at a concentration in a range of from about 2.0 wt % to about 7.0 wt %, based on the total weight of all non-solvents in the polymeric membrane.

31. The polymeric ion selective membrane of claim 25, wherein the at least one monovalent selective ionophore comprises at least one K+ ionophore.

32. The polymeric ion selective membrane of claim 31, wherein the at least one K+ ionophore comprises Valinomycin and/or 2-Dodecyl-2-methyl-1,3-propanediylbis[N-[5′-nitro (benzo-15-crown-5)-4′-yl]carbamate].

33. The polymeric ion selective membrane of claim 25, wherein the plasticizer is dioctyl sebacate when the at least one monovalent selective ionophore is at least one K+ ionophore or at least one Na+ ionophore.

34. A method of minimizing sensor interference in an ion selective electrode sensor, the method comprising the steps of:
providing an ion selective electrode sensor comprising an ion selective membrane, the ion selective membrane having a chemical composition comprising:
a polymer,
a boron-containing salt,
a plasticizer, and
at least one monovalent selective ionophore; and
exposing the ion selective membrane to a cleaning agent comprising benzalkonium;
wherein the ion selective electrode sensor has less than +10.0% induced bias offset upon exposure to the cleaning agent.

35. The method of minimizing sensor interference of claim 34, wherein the ion selective electrode sensor has less than about +5.0% induced bias offset upon exposure to the benzalkonium-containing cleaning agent.

36. The method of minimizing sensor interference of claim 34, wherein the boron-containing salt is provided at a concentration in a range of from about 0.6 wt % to about 1.8 wt %, based on a total weight of all non-solvents in the chemical composition of the membrane.
